(19) United States
(12) Patent Application Publication
(10) Pub. No.: US 2006/0271087 A1
(43) Pub. Date: Nov. 30, 2006

(54) Fixed-volume inflation system for balloon catheters

(75) Inventors: Peter M. Von Dyck, Fernandina Beach, FL (US); John S. Minasi, Amelia Island, FL (US); James G. Schneider, Chesterfield, MO (US); Nick Martino, Fernandina Beach, FL (US); Raymond O. Bodicky, Oakville, MO (US)

Correspondence Address: HUSCH & EPPENBERGER, LLC, 190 CARONDELET PLAZA, SUITE 600, ST. LOUIS, MO, 63105-3441, US

(73) Assignee: Bowel Management Systems, LLC

(21) Appl. No.: 11/137,258

(22) Filed: May 25, 2005

(51) Int. Cl. A61M 29/00

(52) U.S. Cl. 606/192

(57) ABSTRACT

An inflation system (10, 110, 120, 130) for a balloon catheter assembly (12) includes an inflation tube (22), the first end of which is connectable to an inflation fluid reservoir (24, 30) and the second end of which is connectable to and opens into an inflatable cuff (20) on a main catheter (14) of the balloon catheter assembly, to permit the fluid reservoir to be in fluid communication with the cuff. A fluid reservoir is connectable to the inflation tube and is fillable with cuff inflation fluid only to a predetermined, fixed volume, to thereby permit no more than such volume of fluid to be transferred from the reservoir to the cuff during use of the system, to prevent over-inflation of the cuff and thereby avoid potential trauma to the patient.

### BACKGROUND OF THE INVENTION

1. Field of the Invention

The present invention relates, generally, to inflation systems for catheters with balloon-style retention mechanisms, and, more specifically, to a fixed-volume inflation system by which to inflate the retention balloon of an indwelling catheter with a pre-selected fixed volume of inflation media, to thereby prevent over-inflation of the balloon.

2. Related Art

The present invention is considered for use primarily (but not necessarily exclusively) with or as an improvement to known bowel management devices, such as those presently available and marketed by Zassi Medical Evolutions, Inc and Bowel Management Systems, LLC. These systems are described in U.S. Pat. No. 5,569,216 and pending U.S. application Ser. No. 10/225,820, published as US 2004/0039348 on Feb. 26, 2004, the entire disclosures of which patent and application are incorporated herein by reference. The present invention can also be incorporated into other catheter devices having balloon retention mechanisms.

Previously, a variety of catheters used in the body have had balloons or cuffs to hold them in place or to create a seal between the host organ and the outside environment (e.g., Foley catheters, endotracheal tubes). In the majority of these catheters there is no safeguard to prevent the balloon or cuff from being over-inflated by the caregiver, the end result of which is an oversized balloon or cuff that can cause trauma to the organ in which it resides, due to excessive pressure on the surrounding tissues, or even rupture of the cuff.

One example of known medical inflation control devices is an external balloon placed in-line with the inflation circuit of a pulmonary artery catheter. The external balloon is designed to inflate and absorb excess volume from the inflation syringe after an internal balloon contacts the vessel wall. When the catheter tip is in a small pulmonary artery, expansion of the external balloon indicates that the catheter tip is in a noncompliant or excessively small vessel. The in-line external balloon of the known artery catheter does not prevent over-inflation; it just bleeds off excess inflation fluid to the reservoir (external) balloon. This known system achieves the desired result, but is more sensitive to transient environmental changes than is necessary for most indwelling catheters. It is also more sophisticated and expensive than is necessary to achieve the presently desired result; i.e., over-inflation protection. [00051 Another example of known medical inflation control devices are the syringes used to inflate angioplasty balloons. These known syringes incorporate a gauge for monitoring of inflation pressure and are commercially available as, for example, the Monarch line currently from Merit Medical. Such known inflation control syringes do not achieve the presently desired result because the goal of the invention is to prevent over-inflation of the balloon cuff, not necessarily over-pressurization, as the user could easily over-inflate the retention balloon by merely ignoring the pressure gauge readings.

### SUMMARY OF THE INVENTION

By contrast to the known art, it is among the goals and advantages of the present invention to provide a system or device to prevent over-inflation of a catheter retention balloon, which system is simple in construction and use, and incorporates a closed, fixed-volume system so that the user cannot, either inadvertently or intentionally, over-inflate the cuff.

The present invention is also an inexpensive, easy and safe solution to implement. By using proprietary connections (such as those described herein) between the inflation reservoir and the inflation tube, the user cannot inadvertently connect another, inappropriate, infusion device (e.g., a simple luer-tipped syringe) and infuse additional, unnecessary inflation media or other fluid.

A further advantage of the present invention is that diffusion of the inflation media through the balloon membrane does not occur because the balloon membrane is constructed of a non-permeable balloon membrane material and/or the inflation media is a high molecular density substance that cannot diffuse through the membrane. Because the membrane material is non-permeable the system still has the advantage of using standard water based fluids for inflation/expansion purposes. The result of this impermeability is that the catheter retention balloon does not have to be checked repeatedly, thereby saving care-giver time and reducing accidental human errors as they are commonly seen during routine manipulations of the system.

Still further, the new system has the advantage of being volume-regulating, not pressure-regulating, and is therefore not affected (i.e., does not lose volume) by transient pressure changes in the patient's organ (e.g., contraction of the rectum).

Accordingly, in keeping with the above goals and advantages, the present invention is, briefly, an inflation system for a balloon catheter assembly having a main catheter with a first end and a second end. The first end of the main catheter is proximally disposed within a patient in normal use position and has an inflatable cuff disposed thereon, to sealingly retain the main catheter in normal use position within a patient. The second end of the main catheter is disposed distally and external of a patient during normal use position. The system includes an inflation tube, the inflation tube having a first end and a second end, the first end of the inflation tube being connectable to an inflation fluid reservoir and the second end of the inflation tube being connectable to and opening into the cuff on the main catheter, to thereby permit a fluid reservoir to be placed in fluid communication with the cuff. A fluid reservoir is connectable to the inflation tube and is fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient.

The invention is further, briefly, a bowel management assembly having a closed, fixed-volume inflation system. The assembly includes a main catheter for bowel drainage with a first end and a second end, the first end of the main catheter to be disposed within a patient's rectum during use, and the second end of the main catheter to be disposed distally and external of a patient during normal use position. An inflatable and deflatable cuff is connected around the first end of the main catheter, to retain the main catheter in operative position within a patient during use, when the cuff is in an inflated configuration. The closed, fixed-volume inflation system includes an inflation tube, the inflation tube having a first end and a second end, the first end being connectable to an inflation fluid reservoir and the second end being connectable to and opening into the cuff on the main catheter, to thereby permit a fluid reservoir to be placed in fluid communication with the cuff. A fluid reservoir is connectable to the inflation catheter and is fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient.

The invention is still further, briefly, a method of safely maintaining a catheter in a patient. The method includes the following steps: 1) providing an inflation system for balloon catheters having a main catheter with a first end and a second end, the first end of the main catheter having a deflated cuff disposed thereon, an inflation tube connectable to an inflation fluid reservoir, and the cuff so that the fluid reservoir is in fluid communication with the cuff, the fluid reservoir being fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient; 2) inserting the first end of the main catheter into a patient to an extent that the deflated cuff is entirely within the patient and the second end of the main catheter is external of the patient; and 3) causing substantially all of the fluid in the retention reservoir to pass into the cuff via the inflation tube, thus inflating the cuff to maintain the main catheter in normal use position within a patient.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of an example of a known type of bowel management system into which the present invention can be incorporated.

FIG. 2 is schematic side view of the reservoir/bellows portion of the inflation control device of the present invention, shown in the expanded, pre-inflation position, partially broken away.

FIG. 3 is a partial sectional view of the bellows device of FIG. 2, shown in a collapsed/locked position.

FIG. 4 is an enlarged perspective view of an inflation device of the present invention connected to the inflatable cuff of a bowel management system.

FIG. 5A is a schematic perspective view of a fill mechanism for the system of FIG. 4, in the pre-fill configuration and including an optional protective sleeve.

FIG. 5B is a schematic perspective view of the fill mechanism of FIG. 5A, in the collapsed configuration after filling of the system balloon.

FIG. 6A is a schematic longitudinal sectional view of another embodiment of the sleeved fill mechanism, here a bellows shaped fluid reservoir, in the pre-fill position.

FIG. 6B is a schematic longitudinal sectional view of the bellows style fluid reservoir of FIG. 6A in the collapsed, configuration after filling of the cuff is complete.

FIG. 7A is a schematic illustration of another embodiment of the new inflation system including a reservoir disposed internally of the drainage catheter, in the “pre-fill” configuration, prior to inflation of the retention balloon/cuff.

FIG. 7B is a sectional view taken on line 7B - 7B of FIG. 7A.

FIG. 7C is a longitudinal schematic view of the system of FIG. 7A, showing the process of inflation of the cuff.

FIG. 7D is a schematic view of the system of FIG. 7A with the reservoir empty and in a position for waste to drain from the patient via the main catheter.

FIG. 7E is a schematic view of the system of FIG. 7A illustrating a step in optional detachment of the fluid tube from the internal inflation fluid reservoir to initiate cuff deflation in the system.

FIG. 7F is a schematic view of the system of FIG. 7A illustrating further deflation of the cuff.

FIG. 8A is a schematic illustration showing the approximate dimensions of a standard luer lock syringe female fitting.

FIG. 8B is a schematic illustration showing the approximate dimensions of a standard luer lock syringe male fitting.

FIG. 9A is a schematic illustration showing the approximate dimensions of an outlet socket for use in the new system to permit release of inflation fluid from the balloon/cuff.

FIG. 9B is a schematic illustration showing the approximate size of an inlet socket for use in the new system to permit flow of inflation fluid into the balloon/cuff.

FIG. 10A is a longitudinal schematic view, partly in section, of another embodiment of the new inflation system, having an inflation fluid reservoir internal of the main cathetetr of a bowel management system and including separate cuff inflation and deflation tubes.

FIG. 10B is a schematic illustration of the system of FIG. 10A during inflation of the cuff of a bowel management system.

FIG. 10C is a schematic illustration of the general configuration of the system of FIG. 10A during drainage of a use patient's bowel.

FIG. 10D is a schematic illustration of the system of FIG. 10A showing deflation of the cuff via he deflation tube.

FIG. 11A is a longitudinal, broken away view of a portion of the new system including a cuff-fill fitting connecting the inflation fluid reservoir to a fluid tube and including a one-way valve.

FIG. 11B is a longitudinal sectional view of a cuff emptying fitting, including a one-way valve.

Throughout the drawings, like parts are indicated by like element numbers.

### DETAILED DESCRIPTION OF PRACTICAL EMBODIMENTS

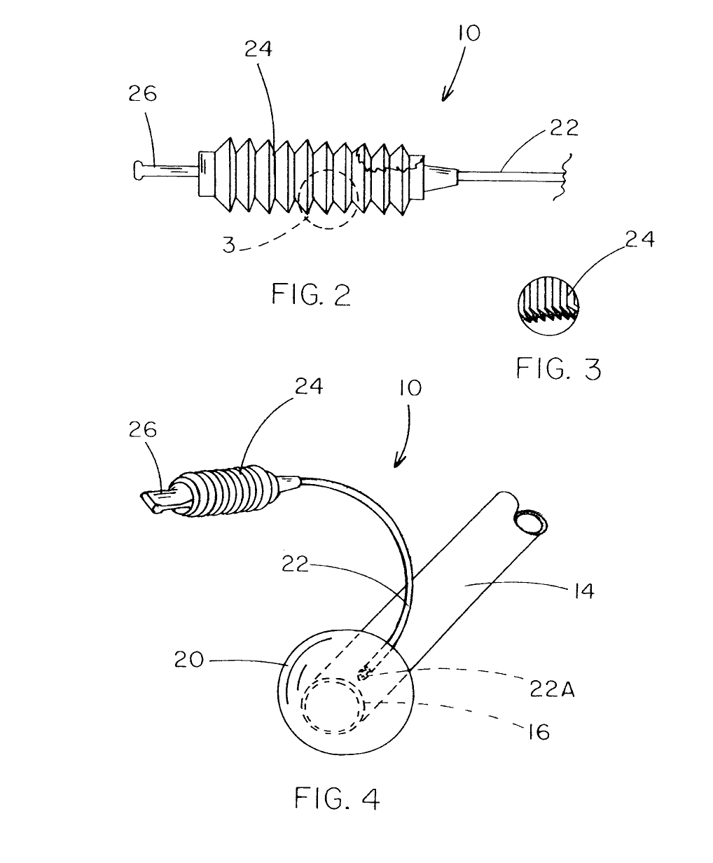

The following description of practical embodiment(s) is merely exemplary in nature and is in no way intended to limit the invention, its application, or uses. It is common to the various useful embodiments of the present invention that they all include a fixed volume reservoir for inflation fluid and a relatively small diameter elastomeric single lumen tube for transferring inflation fluids; i.e. liquids, gels or gases, in and out of a system retention balloon, such as the spherical-shaped cuff style shown in FIGS. 2-4 or some other shaped cuff, such as a more doughnut shaped version, both of which in use are disposed within a patient's rectum. For clarity of the specification the invention is described here relative to such a bowel management system. However, as previously stated, the new inflation system can also apply to other catheters, such as tracheotomy or intravenous catheters, with balloon retention mechanisms that may or may not have an overall precisely spherical shape. For convenience the bolster/retention mechanism of the new system may sometimes be referred to as a “cuff.”

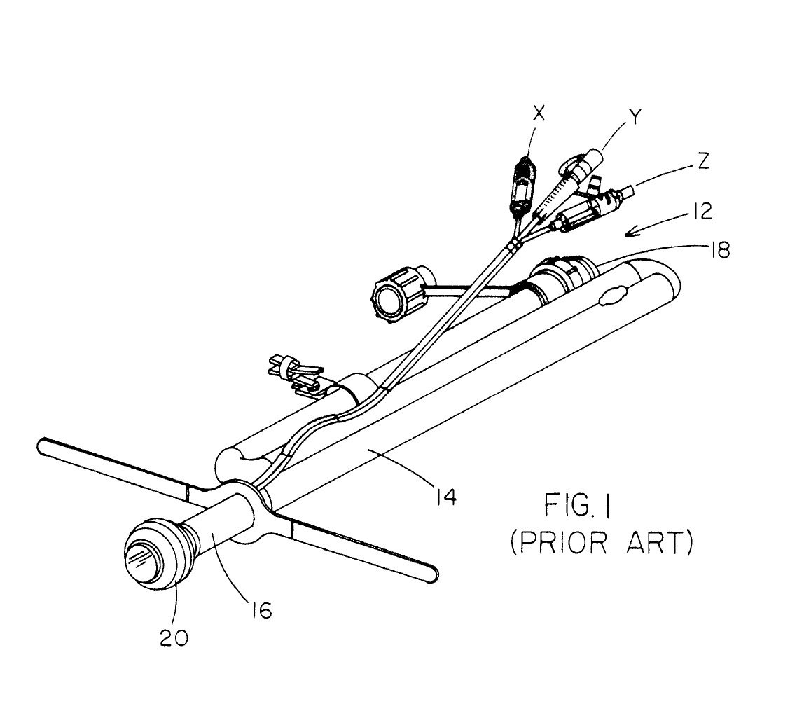

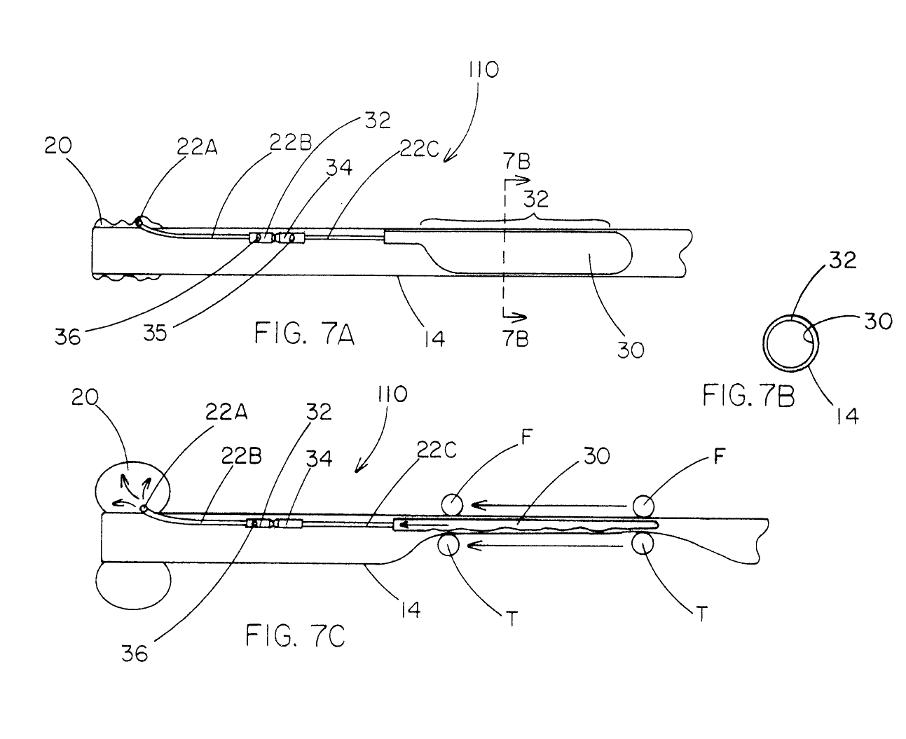

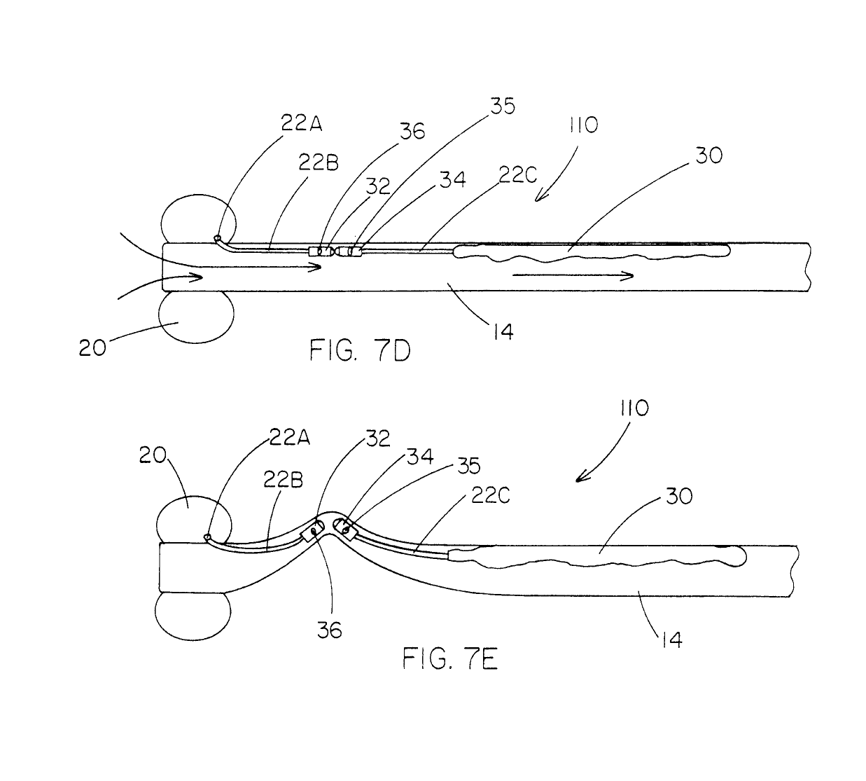

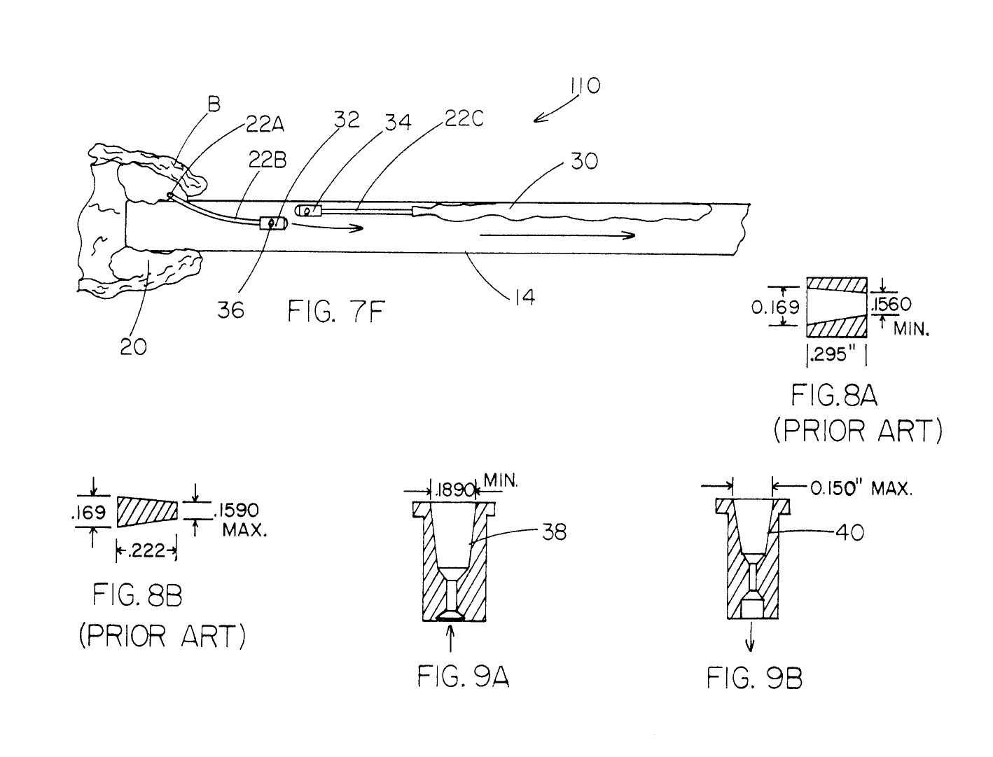

The new closed, fixed-volume inflation system of the present invention is used for example, with a balloon-retained catheter system, for example, the Bowel Management System (“BMS”) 12, as shown in FIG. 1. BMS 12 generally includes a main catheter 14 having patient proximal 16 and distal 18 ends. Patient proximal end 16 of catheter 14 has mounted thereon a cuff or balloon 20, which is collapsed, as seen in FIG. 7A, for ease of insertion of the catheter into the patient's rectum, and then inflated, as seen in FIG. 1, for example, once in the correct position, to hold catheter 14 comfortably within the patient so that drainage of the patient's bowel can be accomplished via the main catheter 14. Cuff 20 may be formed for example, of polyvinylchloride, polyethylene, polyimide, nylon, or polyurethane. Alternatively, the cuff can be formed of a balloon material reinforced with a web material, wherein the balloon material can be, for example, silicone or polyurethane reinforced with a web material of nylon, polyester or other suitable, flexible but strong substance.

A relatively small and preferably single lumen “inflation” tube 22 is provided for inflation of cuff 20 and includes an outlet port 22A at the patient proximal end, exiting to a space between cuff/balloon 20 interior wall and the external wall of a proximal end of the waste catheter tube, the “main” catheter 14. The opposite, patient distal end of inflation tube 22 is connected or connectable to an inflation mechanism of a number of varieties, examples of which are disclosed hereafter.

The patient proximal end of the waste drainage (main) catheter 14 has a relatively large interior diameter, compared to the inflation tube 22, and preferably coaxially penetrates the cuff/balloon 20 to which it is connected, for example by bonding or other suitable means.

Generally speaking, the new inflation system will take one of two overall forms, with either a pre-attached, non-removable inflation fluid reservoir connected externally to a bowel management system catheter, such as 14 in FIG. 4, or with an internal reservoir like that shown in FIG. 7A, for example. The external system can attach to a fill port located at whichever one of the three ports indicated at X,Y,Z, in FIG. 1, which is in fluid communication with cuff 20, or elsewhere, as shown and discussed hereafter. In FIG. 4 the fill port of inflation tube 22 is connected closer to cuff 20 than is shown in FIG. 1. If preferred, an external inflation fluid reservoir can be selectively attachable and removable from the bowel management system or other indwelling catheter system. A number of practical versions of the new close, fixed-volume inflation system for balloon catheters discussed herein are shown in whole or in part in the figures, as described below. Some structural features are common to the alternative configurations of the new inflation system. All configurations can be designed for use with inflation media (“fluid”) with high-molecular density, such as an aqueous solution of polyethylene glycol, for example, that is less likely to diffuse through the balloon membrane than some more well-known inflation media, such as water or ambient air. The cuff balloon membrane material of each embodiment is fabricated in a pre-distended configuration, is compliant, pliable (similar to the existing Bowel Management System cuff) and has a low porosity, at least in regards to the inflation media. Further, the new inflation reservoir has a pre- selected, fixed volume, so that, once filled with inflation fluid (either during manufacturing or by the user), the reservoir cannot be over filled or re-filled. Moreover, each of the described forms of the inflation system is preferably capable of both selectively infusing and withdrawing the inflation media, as desired.

FIG. 2 illustrates one embodiment of the new system, generally designated 10, with an inflation fluid reservoir 24 having an expanded accordion shape when filled. Ideally, the total volume of reservoir 24 and inflation tube 22 is in the range of about 35 cc to about 40 cc of inflation fluid, assuming a cuff 20 of suitable size to retain this volume in an expanded, but not overly expanded state, so as to not be rigid or in danger of bursting when inflated. FIG. 3 illustrates a portion of the compressed pleats of reservoir 24 when the reservoir is manually collapsed to force all but a small residual amount of the inflation fluid therefrom to fill cuff 20. A push/pull tab is indicated at 26 on each end of reservoir 24 and is useful for pushing reservoir 24 closed, or for pulling the reservoir open if desired or necessary. The accordion pleats of reservoir 24 effectively “self lock” the accordion in the closed position of FIG. 3 when fully pushed together.

In the embodiment 10, shown in FIGS. 2-4, the inflation fluid reservoir 24 of system 10 is exterior to catheter 14 and preferably, although not necessarily, permanently connected to inflation tube 22 such that reservoir 24 is in fluid communication with cuff 20, for example, by use of inflation tube 22 connected at one of its two ends to the fluid reservoir 24 and at the other end, preferably internally of the inflation cuff 20 in such a manner, for example by bonding, that it cannot be disconnected from the cuff.

Bellows 24 or other reservoir of the new device can be provided pre-filled by the manufacturer with suitable inflation fluid, or alternatively it can be filled by the patient's caregiver or a health professional prior to use in the patient, but filling must in any case be only to the preselected maximum volume of the reservoir, which is in turn the volume of cuff/balloon 20.

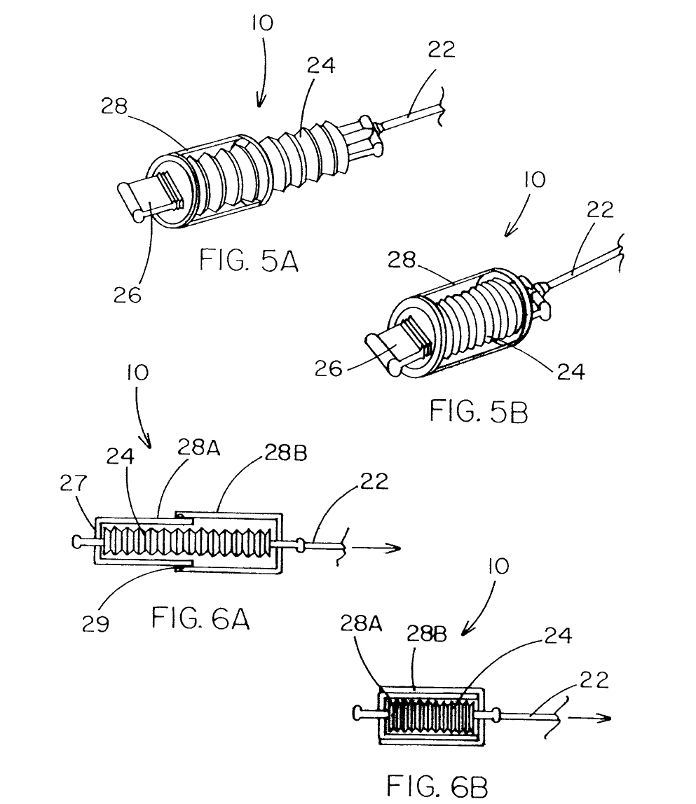

For safety purposes, i.e., to prevent inadvertent compression or puncture of bellows style fluid reservoir 24 a protective sleeve 28 may be included in system 10, such as illustrated in FIGS. 5A, 5B. In these figures, sleeve 28 extends over only part of bellows reservoir 24 when the reservoir is fully inflated, but covers the entire extent of reservoir 24 when in the collapsed, post cuff-filled configuration.

FIGS. 6A, 6B illustrate a variation of the version shown in FIGS. 5A, 5B, wherein the sleeve protector has two portions 28A, 28B sized to fit around the circumference of reservoir 24, but in sliding coaxial relationship to one another, so that as they are forced coaxially toward one another closed ends of the sleeve portions push on corresponding ends of bellows 24 to thereby force fluid within the bellows reservoir out via tube 22 and into cuff/balloon 20, which is not seen in this set of figures. Protective sleeve 28 (or 28A, 28B of FIGS. 6A, 6B) can be maintained in position covering bellows 24 by an annular detenting groove 29 (seen in section in FIG. 6A), on the internal wall of the sleeve, into which a circular base plate 27 of the tab can pressure fit.

It is expected that if bellows reservoir 24 is shipped to the end user in an unfilled state it will be protected from crushing by packaging and no sleeve 28 or other protective device will be necessary, as long as the system is not removed from the shipping package until just before filling and insertion of system 10 to normal use position within a patient. So, the bellows may be shipped disconnected from the catheter. Then, after the user fills the bellows and attaches it to the fluid tube the bellows cannot be disconnected, so that no additional media can be introduced into the cuff.

FIGS. 7A through 7E illustrate another embodiment of the present invention in which the system, generally designated 110 has an intra-luminal reservoir with an internal break- away drainage mechanism. Like the other embodiments described herein, system 110 includes drainage catheter 14 with an inflatable cuff/balloon 20 at a patient proximal end. Internal reservoir 30 is provided to the end user in a pre-filled state, as illustrated in FIG. 7A, containing approximately 35—approximately 40 cc of cuff inflation fluid. For stability of the system, it is preferred that reservoir 30 be connected, as for example by bonding, to an internal point or points on the wall of catheter 14, for example as illustrated at point 32 in FIGS. 7A and 7B.

Reservoir 30 is in fluid communication with cuff 20 via a fluid transfer tube, which as shown is formed of two tube sections 22B, 22C. Tube sections 22b, 22c are selectively releasably connected to each other in fluid-tight relationship by virtue of a break-away conduit connector having sections 32, 34 attached to facing ends of corresponding tubes 22B, 22C, respectively. Drainage catheter 14 is formed of a soft, pliable material, for patient comfort, and also to permit manipulation of fluid reservoir 30 within the catheter. FIG. 7C illustrates by arrows action of a thumb T and finger F which are moved slidably along the length of catheter 14 over the area containing reservoir 30 so as to “milk” inflation fluid from reservoir 30 and thereby forcibly eject the fluid through tube(s) 22B, 22C, through outlet port 22A and into the inflation balloon 20. Break away connector section 34 attached to tube 22c contains a one-way valve 35, for example as at 35 in FIG. 7A, to prevent inflation fluid from backing up from the cuff into reservoir 30.

FIG. 7D indicates by arrows the direction of fecal material flow from the patient via catheter 14 past deflated reservoir 30 toward the patient distal end of catheter 14. Once drainage from the patient is complete, and as illustrated in FIG. 7E, catheter 14 is folded at the intersection of breakaway connector sections 32, 34 until the sections snap away from one another, thus allowing inflation fluid to flow from retention balloon 20 via tube 22B and section 32 into catheter 14. FIG. 7F shows cuff 20 collapsing within the patient's body B as inflation fluid continues to flow out of tube 22C into catheter 14 and out of system 110. Inflation fluid will continue to flow from the balloon 20 as drainage catheter 14 is slowly and gently withdrawn from the patient's rectum.

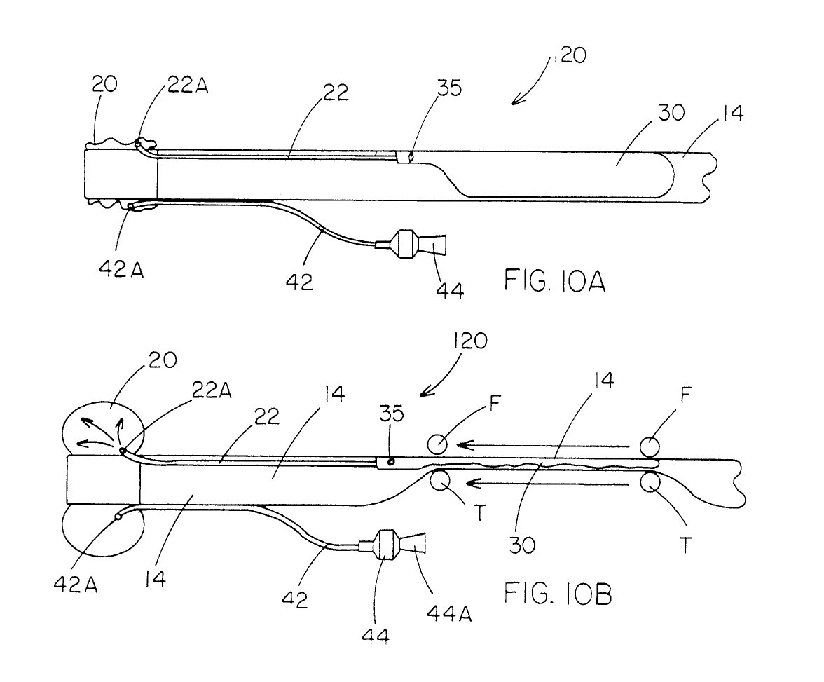

FIG. 8A illustrates the well-known standard dimensions of a conventional luer-lock female connector, of the type commonly used on syringes. FIG. 8B illustrates the well-known standard dimensions of a conventional luer-lock male connector also commonly used on hypodermic syringes, for example. By contrast, FIGS. 9A and 9B show the dimensions of one type of connector designed specifically for use with some embodiments of the new inflation system.

FIG. 9A illustrates dimensions and shape of a one-way/out-only check relief valve 38 which would receive inflation media flowing out of a deflating cuff 20. FIG. 9B schematically illustrates the shape and dimensions for a one-way /in-only check valve 40 connectable to the inflation cuff to permit fluid flow into the cuff, but no exit. This is one example of a suitable connector which is port compatible with the inflation fluid reservoir of the new system and can be used with reservoirs of varying styles, such as a plunger type, bellows (e.g. 24), or bulb, for example. The new input socket is smaller than a standard female luer socket so that a standard male luer tip, sized as illustrated in FIG. 8B, will not be accepted by the new female socket, which is sized as shown at 38 in FIG. 9A. This will prevent inadvertent filling of cuff 20 with something other than the desired inflation fluid, whether instead of or in addition to the fluid provided in a pre-filled reservoir 20. Further, the optional outlet connector sized as at 38, to empty cuff 20, is larger than a standard female luer socket, so a standard male luer tip will be received into a new socket sized as illustrated in FIG. 9A, but the standard tip will not be able to seal and thus will not be able to successfully permit draw of inflation fluid from the cuff. Likewise, a standard luer-type female connector, sized as shown in FIG. 8A, is too large to receive in fluid-tight relationship a new male tip sized as shown in FIG. 9B at 40.

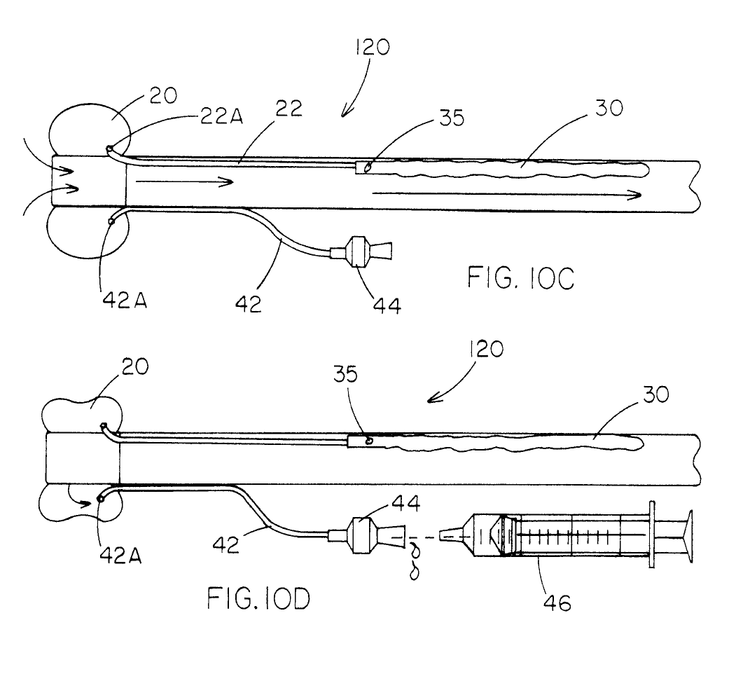

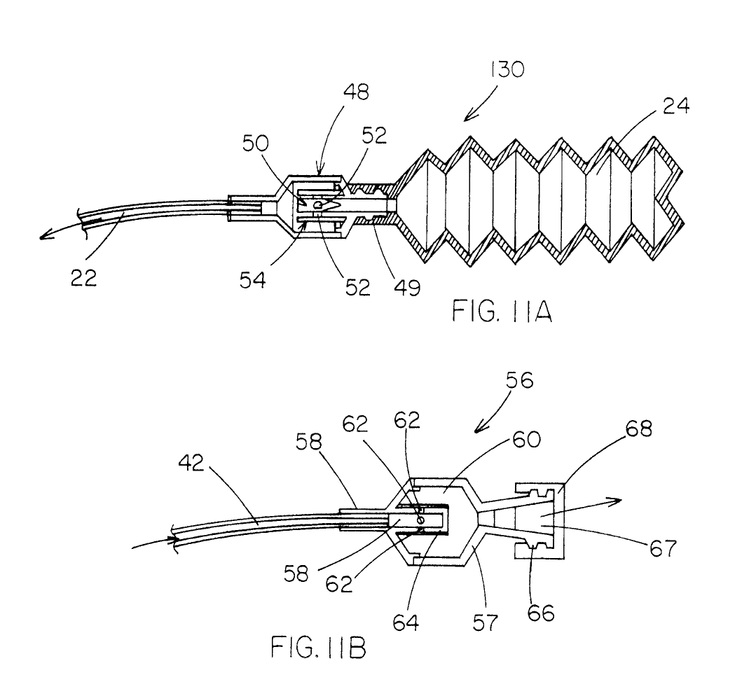

Such new connectors sized as illustrated at 38, 40 could be used in a dual tube system, such as that illustrated in FIGS. 10A through 10D and generally designated 120. However, the embodiment shown in this series of figures preferably is provided with different types of valves. FIG. 10A shows an embodiment of the new system, generally designated 120 in a configuration such as can be useful during the packaging stage. Cuff 20 is mounted at the patient proximal end of drainage catheter 14 in similar manner as in the other embodiments. However, in this case cuff 20 includes an inlet port 22A from fluid tube 22 in addition to an outlet port 42A connecting an outlet tube 42 to cuff 20 to thereby provide fluid communication from the cuff to outside of system 120.

FIG. 10A illustrates system 120 with inflation fluid reservoir 30 pre-filled to the preferred approximate 35 cc to about 40 cc volume. As in the prior embodiments, the collapsible reservoir 30 is preferably blow-molded of thin material and bonded to the interior of catheter 14, as previously described with reference to system 110. A fluid tube 22 connects reservoir 30 to cuff 20 and a one-way valve 35 is disposed either in the neck of reservoir 30, as shown, or in tube 22. Thus, when inflation fluid is milked by a finger F and thumb T, for example, from collapsible reservoir 30 (as indicated in FIG. 10B) the fluid passes through one-way valve 35 into tube 22 and cannot return to the reservoir. Thus, the inflation fluid must exit port 22A into cuff 20 to expand the cuff to no more than the volume of the fluid provided originally in reservoir 30.

FIG. 10C shows reservoir 30 collapsed, after emptying so that it does not block the lumen of catheter 14 and permits fecal mater flow from the patient outwardly through catheter 14. Once the bowel drainage process is complete, inflation fluid in cuff 20 can be removed via a tube 42 which, as shown in FIG. 10D is connected to the cuff at port 42A and terminates preferably in an outlet valve 44, which can be the new version disclosed above for use only with a specially designed withdrawal mechanism, or the valve 44 may be of a standard luer size for fluid removal by a conventional syringe, as indicated in FIG. 10D. When the fluid is removed in this manner system 120 can be carefully withdrawn from the patient's rectum. Alternatively, lacking an independent mechanical removal mechanism, such as syringe 46, simply by carefully withdrawing system 120 from the patient, the pressure of the patient's rectum on the circumference of balloon 20 will cause inflation fluid to be slowly forced out through port 42A until the balloon is collapsed enough to withdraw the entire system. Preferably valve 44 incorporates a one-way out valve that will not permit re-inflation of cuff 20 via valve 44. Alternatively, system 120 can incorporate non-luer compatible connections, such as those previously described, to prevent re-inflation of cuff 20. In this embodiment, it is also preferred that the flaccid reservoir 30 also not be refillable.

FIG. 11A illustrates a filled accordion-style reservoir 24 in fluid communication with tube 22 for filling of a cuff of the system (not seen here) with inflation fluid. It is to be understood that other styles of reservoirs may also be used with this connector. In this embodiment, generally designated 130, there is a fill connector (fitting) 48 attaching tube 22 to reservoir 24. Connector 48 has a substantially cylindrical outer wall which tapers on the patient proximal end to receive an end of tube 22 in fluid-tight relationship. The patient distal end of connector 48 connects to the reservoir 24, preferably by interconnecting non-standard threads with corresponding threads 49 on each of the reservoir and connector 48. A threaded extension of connector 48 connects with reservoir 24, and has at an opposite end a hollow, close-ended stem 50, which extends into a space within connector 48 and defines by the stem wall a number of pressure relief apertures 52. Apertures 52 are covered by a thin elastomeric sleeve 54. Upon application of sufficient pressure to inflation fluid reservoir 24 fluid therein is forced outward from apertures 52 and into the chamber of connector 48 and ultimately out of the connector and into tube 22 to fill cuff 20 (not seen in this view). Inflation fluid cannot flow back into connector 48 because under reverse pressure sleeve 54 would cover apertures 52. Thus connector 48 is essentially a one-way valve which permits inflow of inflation fluid to cuff/balloon 20 but prevents inadvertent loss of inflation fluid volume by back flow to the reservoir.

In use, to perform the balloon-fill procedure, the pre-filled bellows is collapsed, pressurizing the fluid and forcing the elastomeric sleeve away from the ports, thereby allowing passage of the fluid to the balloon. When pressure is released from the bellows valve 48 closes, i.e., sleeve 54 returns to a resting position covering apertures/ports 52. The bellows is then removed and a closure cap (not shown) can be applied to the fill connector if needed or desired. 100621 FIG. 11B illustrates an outflow connector 56 which functions effectively the same as connector 48, but in the opposite direction. Connector 56 has an open ended stem 58 which slidingly connects in fluid-tight relationship to a patient distal end of out flow tube 42. The exterior wall (housing) 57 of connector 56 defines a space 60 into which the opposite end of stem 58 extends, away from the patient and terminates in a closed end. The wall of stem 58 internally of space 60 defines a number of apertures 62 to thereby permit flow of inflation fluid out of cuff 20 (not seen in this view). As in the previously described connector 48, in this connector there is also a thin, pliable elastomeric sleeve 64 which covers apertures 62 unless sufficient cuff deflation pressure is applied to force fluid from the cuff, via tube 42 through the apertures and into space 60. At the patient distal end of connector 56 there an exit port 66 which is provided with an opening 67 and closure 68 for the opening, to preserve balloon fluid volume, if necessary. Fluid opening 67 in port 66 and closure connection 68 may be sized to a standard female luer lock component. A standard luer can be used at this outlet because the one-way valve will block any attempt to refill the balloon cuff 20 through connector 56.

In use, to empty the balloon, a standard 60 cc syringe, for example, is attached to the female luer. The withdrawal of the syringe's plunger causes enough negative pressure to allow the suction force on the fluid to release sleeve 64 from apertures 62, thereby allowing one-way fluid flow from balloon 20. Outflow connector 56 can also act as a pressure relief valve for cuff 20 if the cap of the exit port is removed.

As has been shown, a variety of useful embodiments of the new closed, fixed volume inflation system for balloon catheters are conceived. In addition to the specific examples shown and described, some structural aspects of the embodiments shown can be substituted with others shown or readily apparent to the skilled practitioner in view of this disclosure.

For purposes of further explanation of the new system, the invention can include a two-way valve or clamp mechanism that prevents inflation media from returning to inflation reservoir or inflation tube unless the user specifically intends to empty the cuff/balloon. This would occur, for example, when it is desired to remove a bowel management system from a patient. An example of such a useful valve mechanism would be a pre-attached syringe of known variety, with a stopcock on the end that can be turned to a closed position once the pre-determined volume of media is infused. The media cannot then be withdrawn unless the end user returns the stopcock to its original position and pulls back on the syringe. If preferred, other suitable manipulable or automatic valve mechanisms can be substituted for the stopcock, such as those shown and described herein.

The inflation fluid reservoir can be built into the body waste drain catheter; i.e. so as to be integral with the main catheter and the inflation tube therefor, such as shown in FIGS. 7A-7F, or the reservoir can be external to the drain tube. Examples of the external configurations are illustrated in FIGS. 2-4, 5A-6B and 10A, 10B. If the inflation media reservoir is placed inside the drain tube (catheter 14) it is preferably formed as a pliable bladder configuration, such as that illustrated in FIGS. 7A-7F, so that the fluid reservoir/bladder collapses with drain tube 14 when laid upon by the patient. Alternatively the inflation reservoir can be configured to break away and leave the system, for example, when formed with a connection such as that shown in FIGS. 7A-7F, if not sealed to the inside of catheter 14.

In the latter alternative of the present invention the inflation fluid reservoir is removable from the rest of the system. In that case the reservoir and fluid tube have a proprietary connection with the balloon inflation tube that precludes the attachment of another type of inftision reservoir device (e.g., syringe). For example, the inflation device could be a specially designed syringe with a “keyed” tip to open the fill port by size, as illustrated in FIG. 9B, or otherwise, for example, as by some unique shape. If a syringe is used, with a keyed connection, for example, the syringe can be either pre-filled or limited in volume so that there is no chance of overfilling the retention bolster.

It is further conceived that upon removal or completion of the cuff filling operation an internal mechanism (not shown) shuts off the fill conduit and opens the deflate conduit, in a two lumen system shown in FIGS. 10A-10D. Such an arrangement requires the deflate sequence to occur before the internal mechanism is switched back to the fill position.

The fill port and fill syringe can be keyed to each other by one structural design and the unfill/deflate port and syringe can also be keyed to one another, but to a different design structural design than the first key, so that they cannot be interchanged. In other words, this embodiment is similar in structure to a retractable ballpoint pen. In this embodiment, the filling and emptying of the balloon cuff are accomplished as follows:

In Stage one (filling) a pre-filled fluid system is attached to the connector and displaces an internal element with a one-way fill valve to allow flow of inflation fluid to the balloon. No exit of the fluid is provided here. In Stage two, a static phase, the balloon is filled and the fluid system which causes the internal element to displace into a no-flow static mode is removed. Everything is sealed and the product is used as intended. In Stage three (emptying) a fluid retraction system is attached to the connector, which purposefully permits the internal element to position itself differently that in Stage one, thereby allowing only outward fluid flow from the balloon, via a one-way valve. In the fourth and final, static stage of this version, the balloon is emptied and fluid retraction system is removed which causes the internal element to displace into a no-flow static mode. The system is then ready to go through the sequence again, if desired.

The above-described fill and empty sequence is repeatable, and can be provided with or without a locking mechanism included in the system. If there is a lock out mechanism, that aspect is clearly explained in the system instructions to avoid frustration to the end user. Further safeguards can be provided to prevent manipulations from resulting in system overrides.

Thus it may be seen that the present invention addresses the problem, that accidental or intentional over-inflation and expansion of an inflatable cuff with elastomeric properties can result in patient injury. The goal is accomplished by creating a closed-system of inflation media which can be a gas, such as air, liquid or gel, in communication with a balloon/cuff on the proximal end of the main catheter to selectively retain such catheter proximal end within the patient's rectum during the drainage procedure. The new system limits the amount of inflation media available for use to only the volume that is needed, so that users do not over-inflate the bolster. This is accomplished by providing the required amount of inflation media in a pre-filled reservoir that is connected to the retention and sealing balloon/cuff of the subject system and is of such volume to properly fill the bolster to an inflated comfortable state, which is effective for retaining the catheter proximal end within the patient, but not inflating so far that there is danger of the cuff becoming rigid or rupturing from being overfilled.

The operator simply deploys the pre-filled reservoir by squeezing inflation media from the reservoir through a transport tube and into the cuff. The media is then trapped using a clamp or other suitable valve. Deflation is accomplished by releasing the clamp or opening the valve and applying suction or traction in or around the balloon/cuff. The new system of course requires that the inflation media not be able to diffuse out of the balloon/cuff over time. This can be accomplished by using non-porous cuff materials with a typical inflation media such as water or saline, normal semi-porous balloon/cuff materials with viscose, high molecular density inflation media or a combination of the non-porous balloon/cuff material and the viscose, high molecular density inflation media.

As various modifications could be made to the exemplary embodiments, as described above with reference to the corresponding illustrations, without departing from the scope of the invention, it is intended all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An inflation system (10, 10, 120, 130) for a balloon catheter assembly (12) having a main catheter (14) with a first end (16) and a second end (18), the first end of the main catheter being proximally disposed within a patient in normal use position and having a cuff (20) disposed thereon, to maintain the main catheter in normal use position within a patient, and the second end of the main catheter being disposed distally and external of a patient during normal use position; the system comprising:
an inflation tube (22), the inflation tube having a first end and a second end, the first end of the inflation tube being connectable to an inflation fluid reservoir (24, 30) and the second end of the inflation tube being connectable to and opening into the cuff on the main catheter, to thereby permit a fluid reservoir to be placed in fluid communication with the cuff; and
a fluid reservoir (24, 30) connectable to the inflation tube, the fluid reservoir being fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient.

2. The inflation system of claim 1, and further comprising inflation fluid within the inflation fluid reservoir, the inflation fluid having a sufficiently high molecular density to prevent the inflation fluid from passing through the membrane material of the catheter cuff.

3. The inflation system of claim 2, wherein the inflation fluid is aqueous polyethylene glycol.

4. The inflation system of claim 1, wherein the fluid reservoir is disposed externally of the main catheter.

5. The inflation system of claim 4, wherein the fluid reservoir has a bellows shape.

6. The inflation system of claim 5, wherein the reservoir is formed of a plurality of adjacent pleats so that longitudinal compression of the bellows-shaped reservoir pushes the pleats together and thereby secures the bellows in a closed and substantially empty configuration.

7. The inflation system of claim 6, and further comprising a sleeve mounted outside of and substantially coaxially to the bellows-shaped fluid reservoir to prevent inadvertent compression of the reservoir and resultant inflation of the cuff.

8. The inflation system of claim 7, wherein the protective sleeve around the bellows-shaped reservoir is formed of two coaxially disposed sections, sized so that one section can be slidably moved into the other to force an end of the reservoir toward an opposite end of the reservoir to thereby push fluid within the bellows-shaped reservoir into the cuff via the inflation tube.

9. The inflation system of claim 5, wherein the bellows-shaped fluid reservoir is provided with a tab on one end of the bellows to facilitate pushing the bellows closed to force fluid from the bellows via the inflation tube and into the cuff, and to facilitate pulling the bellows open to withdraw fluid from the cuff and thereby collapse the cuff and to thereby permit removal of the main catheter from the patient.

10. The inflation system of claim 4, wherein the fluid reservoir is a fixed volume syringe.

11. The inflation system of claim 10, wherein the inflation tube extends externally of the main catheter and is provided on a free end thereof with a non-standard female fitting and further wherein the fluid reservoir syringe has a non-standard male fitting which interlocks in fluid-tight relationship with the non-standard female fitting of the inflation tube to thereby provide an inflation mechanism which cannot be inadvertently filled or over-filled with fluid from any standard syringe.

12. The inflation system of claim 1, wherein the cuff inflation fluid reservoir is disposed internally of the main catheter.

13. The inflation system of claim 12, wherein the cuff inflation fluid reservoir is a collapsible bladder that can be emptied by milking action to force fluid from the bladder into the cuff via the inflation tube.

14. The inflation system of claim 12, wherein the cuff inflation fluid reservoir is connected in part, to an internal wall of the main catheter.

15. The inflation system of claim 1, and further comprising at least one valve within the inflation fluid tube to permit control of fluid flow direction within the fluid tube.

16. The inflation system of claim 15, wherein the inflation tube is formed of a first tube section and a second tube section.

17. The inflation system of claim 16, wherein the first tube section and the second tube section are coaxially joined by adjacent portions of a break-away connector, the first tube section extending from the fluid reservoir and the second tube section extending to the cuff so that when the tube sections are connected the fluid reservoir is in fluid communication with the cuff.

18. The inflation system of claim 17, wherein the break-away connector of the first tube section has a one-way valve which permits fluid flow only away from the fluid reservoir.

19. The inflation system of claim 17, wherein the break-away connector of the second tube section has a two-way valve which permits fluid flow into and away from the cuff, to thereby permit fluid to flow out of the cuff and into a lumen of the main catheter after separation of the adjacent portions of the break-away connector.

20. The inflation system of claim 1, and further comprising a one-way valve disposed at the connection of the inflation fluid reservoir and the inflation tube, to thereby ensure that once fluid is introduced from the system into the cuff on a catheter, that the fluid cannot back flow to any significant extent and cause the cuff to collapse.

21. The inflation system of claim 20, and further comprising a deflation tube, the deflation tube having a first end and a second end, the first end of the deflation tube being fixed in fluid communication with the cuff and the second end of the deflation tube having an outlet port to permit exit of inflation fluid from the cuff to permit removal of the cuff from the patient.

22. The inflation system of claim 21, wherein the outlet port includes a one-way valve to prohibit re-inflation or over-inflation of the cuff via the outlet tube.

23. The inflation system of claim 22, wherein the outlet port is provided with a standard luer lock female fitting to permit removal of the inflation fluid with a conventional syringe.

24. The inflation system of claim 23, wherein the fixed volume fluid reservoir has an outlet formed with a non-standard fitting and further comprising a connector mounted on the patient distal end of the fluid tube and having a one-way valve therein which permits fluid flow only to the fluid tube toward the cuff on the main catheter, the connector having a non-standard fitting which interconnects with the non-standard fitting of the reservoir, to thereby ensure that fluid cannot be introduced into the system by any other mechanism.

25. The inflation system of claim 24, wherein system includes a fluid outlet tube having one end in fluid communication with the cuff, and another end to which is connected an outflow connector including a one-way outflow valve.

26. The inflation system of claim 25, wherein the outflow connector is provided with an end cap to ensure closure of the system.

27. The inflation system of claim 26, wherein the outflow connector is provided with a standard luer lock fitting to permit connection of a conventional syringe for withdrawal of fluid from the system.

28. A bowel management assembly having a closed, fixed-volume inflation system, the assembly comprising:
a main catheter (14) for bowel drainage with a first end (16) and a second end (18), the first end of the main catheter to be disposed within a patient's rectum during use, and the second end of the main catheter to be disposed distally and external of a patient during normal use position
an inflatable and deflatable cuff (20) connected around the first end of the main catheter, to maintain the main catheter in operative position within a patient during use, when the cuff is in an inflated configuration; the system comprising:
an inflation tube (22), the inflation tube having a first end and a second end, the first end of the inflation tube being connectable to an inflation fluid reservoir and the second end of the inflation tube being connectable to and opening into the cuff on the main catheter, to thereby permit a fluid reservoir to be placed in fluid communication with the cuff; and
a fluid reservoir (24, 30) connectable to the inflation catheter, the fluid reservoir being fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient.

29. The bowel management assembly of claim 28, wherein the catheter cuff is formed of a membranous material having a sufficiently low porosity as to prevent the inflation fluid from passing through the cuff membrane.

30. The assembly of claim 28, wherein the catheter cuff is formed of a material selected from the group consisting of polyvinylchloride, polyethylene, polyimide, nylon, and polyurethane.

31. The assembly of claim 28, wherein the catheter cuff is formed of a balloon material reinforced with a web material.

32. The assembly of claim 31, wherein the material of the balloon cuff is selected from the group consisting of silicone and polyurethane.

33. The assembly of claim 31, wherein the web material is selected from the group consisting of nylon and polyester.

34. A method of safely retaining a catheter in a patient, the method comprising: providing an inflation system for balloon catheters having a main catheter (14) with a first end and a second end, the first end of the main catheter having a deflated cuff (20) disposed thereon, an inflation tube connectable to an inflation fluid reservoir, and the cuff so that the fluid reservoir is in fluid communication with the cuff, the fluid reservoir (24, 30) being fillable with cuff inflation fluid only to a predetermined, fixed volume, to thereby permit no more than such predetermined, fixed volume of fluid to be transferred from the fluid reservoir to the cuff during use of the system, to prevent over-inflation of the cuff and thereby avoid potential trauma to the patient;
inserting the first end of the main catheter into a patient to an extent that the deflated cuff is entirely within the patient and the second end of the main catheter is external of the patient; and
causing substantially all of the fluid in the inflation fluid reservoir to pass into the cuff via the inflation tube, thus inflating the cuff to maintain the main catheter in normal use position within a patient.
